(19) United States
(12) Patent Application Publication
(10) Pub. No.: US 2025/0319018 A1
(43) Pub. Date: Oct. 16, 2025

(54) DRUG FORMULATIONS FOR EXTENDED DRUG DELIVERY

(71) Applicant: Mott Corporation, Farmington, CT (US)

(72) Inventor: Alex Hill, Manchester, CT (US)

(21) Appl. No.: 19/177,101

(22) Filed: Apr. 11, 2025

Related U.S. Application Data
(60) Provisional application No. 63/633,367, filed on Apr. 12, 2024.

(51) Int. Cl. A61K 9/00

(52) U.S. Cl. CPC A61K 9/0024

(57) ABSTRACT

A drug delivery assembly, having a housing that defines an interior space, with first and second closures at first and second ends, a divider wall between the first and second ends, a first compartment located at the first end, between the first closure and the divider wall, a second compartment located at the second end, between the divider wall and the second closure; the divider wall has a first porous membrane, that provides fluid communication between the first and second compartments; and the second closure has a second porous membrane, the second porous membrane provides fluid communication between the second compartment and an area external to the housing; and a first drug formulation disposed within the first compartment, the first drug formulation has a first drug and a first excipient, the second compartment including a second drug formulation that is the same as or different from the first drug formulation.

### CROSS REFERENCE

This application claims priority to U.S. 63/633,367, filed on Apr. 12, 2024, the entire contents of which is incorporated herein by reference.

### FIELD OF THE DISCLOSURE

The present disclosure relates to drug formulations, drug delivery assemblies for extended drug delivery and/or tunability and systems/methods for utilizing and fabricating the drug delivery assemblies and, more particularly, to single or dual compartment, and single or dual porous membrane based (e.g., porous zinc membrane based) drug delivery assemblies for extended drug delivery (e.g., via passive diffusion) and/or tunability.

### BACKGROUND OF THE DISCLOSURE

In general, some drug formulations and drug delivery assemblies or the like are known.

An interest exists for improved drug formulations, drug delivery assemblies, and related methods of use.

Opportunities for improvement are addressed and/or overcome by the assemblies, methods and devices of the present disclosure.

### BRIEF SUMMARY OF THE DISCLOSURE

The present disclosure provides advantageous drug formulations, drug delivery assemblies for extended drug delivery and/or tunability, and improved systems/methods for utilizing and fabricating the drug delivery assemblies. More particularly, the present disclosure provides single or dual compartment, and single or dual porous membrane based (e.g., porous zinc membrane based) drug delivery assemblies for extended drug delivery (e.g., via passive diffusion) and/or tunability.

More specifically, disclosed is a drug delivery assembly, including: a housing that extends from a first end to a second end, wherein the housing defines an interior space, with a first closure at the first end, a second closure at the second end, and a divider wall between the first end and the second end, such that the housing further defines: a first compartment located at the first end of the housing, between the first closure and the divider wall, and a second compartment located at the second end of the housing between the divider wall and the second closure, wherein: the divider wall includes a first porous membrane, or defines a first aperture that includes the first porous membrane, wherein the first porous membrane provides fluid communication between the first compartment and the second compartment; and the second closure includes a second porous membrane, or defines a second aperture that includes the second porous membrane, wherein the second porous membrane provides fluid communication between the second compartment and an area that is external to the housing; and a first drug formulation disposed within the first compartment, the first drug formulation including a first drug and a first excipient, and the second compartment includes a second drug formulation that is the same as or different from the first drug formulation.

In addition to one or more aspects of the assembly, or as an alternative, the first drug is present in a particulate form, and wherein the first drug is formed as a microparticle or a nanoparticle.

In addition to one or more aspects of the assembly, or as an alternative, the first porous membrane has a lower drug permeability than the second porous membrane.

In addition to one or more aspects of the assembly, or as an alternative, the first drug is encapsulated in a degradable shell that is formed of a polymer or a gel.

In addition to one or more aspects of the assembly, or as an alternative, the first drug formulation includes a non-polar compound immiscible in water; and the second compartment includes a polar solvent miscible in water.

In addition to one or more aspects of the assembly, or as an alternative, the non-polar compound is one or more of: liquid paraffin; a lipid; synthetic oil; or natural oil; or the polar solvent is water.

In addition to one or more aspects of the assembly, or as an alternative, the first excipient includes one or more of: an emulsion; a colloidal suspension; a surfactant; a perfluorinated compound; and a polymer including a fluoropolymer and nanocellulose.

In addition to one or more aspects of the assembly, or as an alternative, the second drug formulation includes one or more of a second drug and a second excipient, wherein the second drug is the same as or different from the first drug, and the second excipient is the same as or different from the first excipient; and wherein the first drug is provided in a first drug concentration in the first drug formulation and the second drug is provided in a second drug concentration in the second drug formulation, wherein the second drug concentration is the same as or different from the first drug concentration.

In addition to one or more aspects of the assembly, or as an alternative, the second drug is the same as the first drug; and the second drug concentration is less than the first drug concentration.

In addition to one or more aspects of the assembly, or as an alternative, the second drug formulation includes one or more of: a second drug that includes one or more of: a particulate form, wherein the second drug is formed as a microparticle or a nanoparticle; or a degradable shell in which the second drug is encapsulated, wherein the degradable shell is formed of a polymer or a gel; or a second excipient that includes one or more of: an emulsion; a colloidal suspension; a surfactant; a perfluorinated compound; or a polymer including a fluoropolymer and nanocellulose.

Further disclosed is a method of preparing the drug delivery, including: obtaining a first drug formulation that includes a first drug and a first excipient, wherein the first drug is present in a particulate form; obtaining a second drug formulation that is the same as or different from the first drug formulation; filling the first drug formulation within a first compartment of a housing, wherein the first compartment is separated from a second compartment of the housing by a first porous membrane, and the second compartment is separated from an area external to the housing by a second membrane; and filling the second compartment with the second drug formulation, wherein: the housing extends from a first end to a second end, and defines an interior space, with a first closure at the first end, a second closure at the second end, and a divider wall between the first end and the second end, wherein that the housing defines: the first compartment located at the first end of the housing, between the first closure and the divider wall, and the second compartment located at the second end of the housing between the divider wall and the second closure, and wherein: the divider wall includes the first porous membrane, or defines a first aperture that includes the first porous membrane, wherein the first porous membrane provides fluid communication between the first compartment and the second compartment; and the second closure includes the second porous membrane, or defines a second aperture that includes the second porous membrane, wherein the second porous membrane provides fluid communication between the second compartment and an area that is external to the housing.

In addition to one or more aspects of the method, or as an alternative, the first drug is formed as a microparticle or a nanoparticle; and

In addition to one or more aspects of the method, or as an alternative, the first porous membrane has a lower drug permeability than the second porous membrane.

In addition to one or more aspects of the method, or as an alternative, the first drug is encapsulated in a degradable shell that is formed of a polymer or a gel.

In addition to one or more aspects of the method, or as an alternative, the first drug formulation includes a non-polar compound immiscible in water; and the second compartment includes a polar solvent miscible in water.

In addition to one or more aspects of the method, or as an alternative, the non-polar compound is one or more of: liquid paraffin; a lipid; synthetic oil; or natural oil; or the polar solvent is water.

In addition to one or more aspects of the method, or as an alternative, the first excipient includes one or more of: an emulsion; a colloidal suspension; a surfactant; a perfluorinated compound; and a polymer including a fluoropolymer and nanocellulose.

In addition to one or more aspects of the method, or as an alternative, the second drug formulation includes one or more of a second drug and a second excipient, wherein the second drug is the same as or different from the first drug, and the second excipient is the same as or different from the first excipient; and the first drug is provided in a first drug concentration in the first drug formulation and the second drug is provided in a second drug concentration in the second drug formulation, wherein the second drug concentration is the same as or different from the first drug concentration.

In addition to one or more aspects of the method, or as an alternative, the second drug is the same as the first drug; and the second drug concentration is less than the first drug concentration. In addition to one or more aspects of the method, or as an alternative, the second drug formulation includes one or more of: a second drug that includes one or more of: a particulate form, wherein the second drug is formed as a microparticle or a nanoparticle; or a degradable shell in which the second drug is encapsulated, wherein the degradable shell is formed of a polymer or a gel; or a second excipient that includes one or more of: an emulsion; a colloidal suspension; a surfactant; a perfluorinated compound; or a polymer including a fluoropolymer and nanocellulose.

The particulate form of the drug can be a microparticle including the drug and an optional excipient, a nanoparticle including the drug and an excipient, the drug encapsulated by a polymer, a lipid nanoparticle, or a combination thereof.

The drug particle can be present in a solution including soluble drug.

The particulate form provides extended release of the drug compared to the drug when dissolved.

The above described and other features are exemplified by the following figures and detailed description.

Any combination or permutation of embodiments is envisioned. Additional advantageous features, functions and applications of the disclosed assemblies, methods and devices of the present disclosure will be apparent from the description which follows, particularly when read in conjunction with the appended figures. All references listed in this disclosure are hereby incorporated by reference in their entireties.

### BRIEF DESCRIPTION OF THE DRAWINGS

The following figures are exemplary embodiments wherein the like elements are numbered alike.

Features and aspects of embodiments are described below with reference to the accompanying drawings, in which elements are not necessarily depicted to scale.

Exemplary embodiments are further described with reference to the appended figures. It is to be noted that the various features, steps, and combinations of features/steps described below and illustrated in the figures can be arranged and organized differently to result in embodiments which are still within the scope of the present disclosure. To assist those of ordinary skill in the art in making and using the disclosed assemblies, methods and devices, reference is made to the appended figures, wherein:

FIG. 1 is a side cross-sectional view of an exemplary drug delivery assembly, according to the present disclosure.

FIG. 2 is a modeled delivery profile of the drug delivery assembly of FIG. 1 and for an exemplary drug.

FIG. 3 is a modeled release profile of drug showing percent released over time for an exemplary drug delivery assembly according to the present disclosure.

FIG. 4 is a side cross-sectional view of another exemplary drug delivery assembly, according to the present disclosure.

FIG. 5 is a side view of another exemplary drug delivery assembly, according to the present disclosure.

FIG. 6 is an exploded partial view of the drug delivery assembly of FIG. 5.

FIG. 7 is an exploded side view of the drug delivery assembly of FIG. 5.

FIG. 8 is a side perspective view of the drug delivery assembly of FIG. 5.

FIG. 9 is a cross-sectional side view of another exemplary drug delivery assembly, according to the present disclosure.

FIG. 10 is a side perspective view of another exemplary drug delivery assembly, according to the present disclosure.

FIG. 11 is an exploded side view of the drug delivery assembly of FIG. 10, and showing the bottom port area in a side cross-sectional view.

FIG. 12 is a cross-sectional side view of another exemplary drug delivery assembly, according to the present disclosure.

FIG. 13 is a flowchart showing a method of preparing the drug delivery assembly, e.g., prior to implanting in a patient, according to an embodiment.

### DETAILED DESCRIPTION OF THE DISCLOSURE

The exemplary embodiments disclosed herein are illustrative of advantageous drug formulations, drug delivery assemblies, and systems of the present disclosure and methods/techniques thereof. It should be understood, however, that the disclosed embodiments are merely exemplary of the present disclosure, which may be embodied in various forms. Therefore, details disclosed herein with reference to exemplary drug formulations, drug delivery assemblies and associated processes/techniques of assembly and use are not to be interpreted as limiting, but merely as the basis for teaching one skilled in the art how to make and use the advantageous drug formulations, drug delivery assemblies and/or alternative drug delivery assemblies of the present disclosure.

Disclosed herein are advantageous drug formulations, drug delivery assemblies, and related methods of fabrication and use thereof.

The present disclosure provides improved drug formulations, drug delivery assemblies for extended drug delivery (e.g., via passive diffusion) and/or provides tunability, and improved systems/methods for utilizing and fabricating the drug delivery assemblies.

More particularly, the present disclosure provides single or dual compartment, and single or dual porous membrane based (e.g., porous zinc membrane based) drug delivery assemblies for extended drug delivery (e.g., via passive diffusion) and/or to provide tunability, i.e., drug delivery where the release of the drug can be adjusted to a desired regimen.

Referring now to the drawings, like parts are marked throughout the specification and drawings with the same reference numerals, respectively. Drawing figures are not necessarily to scale and in certain views, parts may have been exaggerated for purposes of clarity.

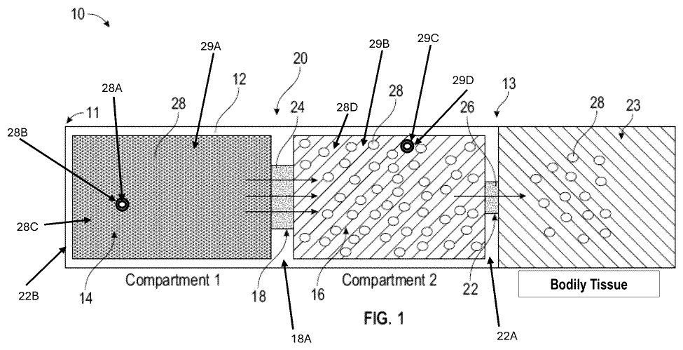

As shown in FIG. 1, there is illustrated a drug delivery assembly 10 usable with the drug formulations disclosed herein.

Exemplary drug delivery assembly 10 takes the form of a dual compartment and dual porous membrane based (e.g., porous zinc membrane based) drug delivery assembly 10 for extended drug 28 delivery (e.g., via passive diffusion) and/or tunability or the like, although the present disclosure is not limited thereto.

As shown in FIG. 1, drug delivery assembly 10 includes a housing 12 that extends from a first end 11 to a second end 13. In exemplary embodiments, the housing 12 is substantially tubular or substantially cylindrical, although the present disclosure is not limited thereto. Rather, it is noted that housing 12 can take a variety of shapes and/or forms, including a form wherein a diameter of the housing is varied.

Housing 12 can be fabricated from a variety of materials. For example, housing 12 can be fabricated from a biocompatible metal (e.g., magnesium, zinc, titanium, iron, stainless steel, or an alloy thereof). Housing 12 can also be fabricated from a biocompatible polymer, e.g., a poly(meth) acrylate or copolymer thereof, a polyurethane, a polyether ketone, or the like; or a biodegradable polymer (e.g., a polyester such as a polycaprolactone, polylactide (e.g., poly (DL-lactide (PLA) or poly (L-lactide) (PLLA), or other isomers and copolymers of lactide), a polyglycolic acid, or a copolymer thereof, a polysaccharide, or the like. Where the housing is biodegradable, it is preferably selected to have a slower biodegradability than any biodegradable substance associated with drug 28, which is described below. In an embodiment, the housing can be fabricated from a material such as zinc, iron, magnesium, titanium, metal alloys, polylactic acid, poly (lactic-co-glycolic acid), or a combination thereof. The housing can be solid, or can be porous, or a combination of solid and porous, to allow the diffusion of a drug 28 as described below through the housing. In an embodiment, all of the housing is solid, except at the portion defining the openings. In an embodiment, if all or a portion of the housing is porous, it has a porosity that is less than a porosity of the porous membranes as described below.

In non-limiting examples, housing 12 takes the form of a tube shape, with an overall length of about 0.5 to about 25 cm, preferably about 0.5 to about 10 cm, more preferably between about 1 to about 5 cm. Housing 12 can have a diameter between about 1 to about 25 mm, preferably between about 2 to about 5 mm. The housing 12 can have a wall thickness between about 1 to about 5 mm. It is noted that the diameter of the housing can be between about 3 to about 7 mm, and the length of the housing can be about between about 3 to about 12.5 mm.

Exemplary housing 12 defines a first compartment 14 and a second compartment 16, with a first opening 18 in housing 12 in communication with the first and second compartments 14, 16. The first opening 18 is located at a position 20 (e.g., an intermediate position 20) between the first and second ends 11, 13 of housing 12.

A second opening 22 in the housing 12 is positioned at the second end 13 of the housing 12 of the assembly 10, in an end wall (or plate) 12C, as shown in FIG. 1. The second opening 22 may have a circular cross section with a smaller area than the first opening 18, in a non-limiting embodiment. In general, the second opening 22 can be in fluid communication with an area 23 that is external to the housing 12 of the assembly 10 (e.g., an area 23 such as, for example, the surrounding tissue of a body of a patient, test subject, or the like, after the assembly 10 is positioned in the body). The patient can be a human or animal in need of the drug. The test subject can be a human or animal.

In exemplary embodiments, a first porous membrane 24 is positioned in the first opening 18, and a second porous membrane 26 is positioned in the second opening 22. In one embodiment, the first porous membrane 24 has a lower drug permeability than the second porous membrane 26.

The first porous membrane 24 can be attached and/or bonded relative to the first opening 18 of housing 12 and that the second porous membrane 26 can be attached and/or bonded relative to the second opening 18 of housing 12 via various attachment or bonding methods (e.g., sintering bonding, adhesive, press-fit, etc.).

FIG. 1 shows a cross section of housing 12 of assembly 10 (e.g., tubular assembly 10), with the assembly 10 having first compartment 14 in fluid communication with second compartment 16 via first porous membrane 24 positioned in first opening 18, and with the assembly 10 having second compartment 16 in fluid communication with area 23 via second porous membrane 26 positioned in second opening 22. Each interior cavity of first and/or second compartments 14, 16 can be filled or partially filled with a drug (or first drug) 28.

Drug 28 can be present as a solution including the drug 28 in dissolved form, or a first drug formulation 29A (for simplicity, a formulation), e.g., a suspension, including the drug 28 in particulate form 28A, a gel including the drug 28, a slow release composition including the drug 28, or the like, or a combination thereof. In an embodiment, the drug is provided in dissolved or particulate form in a solution (e.g., water), and fills the compartment.

When present as particles, e.g., as a salt, the drug can be partially soluble in, e.g., water, and provided extended release of the dissolved drug over time.

In other embodiments, the drug is provided in other forms, such as reversibly attached to a gel or solid support (in dissolved or particulate form), present in a degradable (e.g., a partially to fully dissolvable) matrix such as a particle, gel or solid support, encapsulated in one or more degradable shells 28B, or a combination thereof. Such forms are known to those of skill in the art and are useful to provide even greater tunability of drug release. One or more adjuvants (e.g., a salt, pH adjusting agent, or the like) as is known in the art can be present in addition to the drug.

The functionality of the medication can be improved by tuning (i.e., adjusting) the release profile of the medication, which can in turn be adjusted by use of certain drug formulations. These drug formulations (and the others described generally above) can be especially useful with challenging drug release targets. The formulations can provide one or more, or all of a longer release window, improved drug stability, or a combination thereof.

In an embodiment, the drug is provided in a particulate form, e.g. in an aqueous formulation including an excipient 28C (for example a co-solvent, pH adjusting agent, or the like) and particles including the drug and an excipient. For example, the particle including the drug can include a biodegradable excipient to regulate the release of the drug from the particle.

In an embodiment, the particulate form of the drug can be present in a solution including the dissolved drug, e.g., as a saturated solution of the drug. The upper limit on drug capacity within the drug delivery assembly can be related, e.g., proportional, to a solubility limit of the drug in the drug formulation, and an overall volume of the assembly.

By including particles of the drug (i.e., as an insoluble salt) in combination with an initially drug-saturated formulation within the assembly cavity, higher loadings of the drug can be achieved. For the initial period during which the insoluble salt is present, which may be, for example, about 1 to about 3 months, a concentration of drug within the solution can be saturated. At the time at which the salt is fully dissolved, the concentration within the solution can decrease as the drug is delivered via passive diffusion. In addition to the added benefit of higher solids loading, a rate of delivery of the drug out of the assembly can be maintained as there may not be a decay in concentration/mass transport potential.

The particle can be a microparticle having a dimension (for example, a largest average dimension or a largest dimension) of between about 1 μm (or greater than 1 μm) to about 1,000 μm (or less than 1,000 μm), such as between about 2 μm to about 900 μm, or between about 2 μm to about 500 μm, or between about 2 μm to about 250 μm. The particle can be a nanoparticle having a dimension (for example, a largest average dimension or a largest dimension) of between about 1 nm (or greater than 1 nm) to about 1,000 nm (or less than 1,000 nm), such as between about 2 to about 900 nm, or between about 2 nm to about 500 nm, or between about 2 nm to about 250 nm, or the like. The maximum size of the particle can be selected to be easily loaded into the drug delivery devices by, for example, a syringe, without clogging the syringe. Otherwise, the size and amount of the particle population is selected to provide the desired release profile. For example, use of a bimodal distribution of sizes can provide a bimodal release profile. The particle size can be selected to retain the particles within the compartment, or to allow release of the particle by, e.g., passive diffusion.

In another embodiment where the drug is encapsulated by (e.g., suspended in) one or more polymer spheres within the compartment, the spheres can be packed into the compartment, suspended in an aqueous solvent, or suspended in a polymer matrix, e.g., a biodegradable gel. The polymer used in the spheres can help to protect the drug from degradation from excipients, ambient temperature, lack of innate stability, and/or degradation from the solvent. The spheres can degrade, e.g., slowly, releasing drug into the compartment, which would achieve a goal of reducing a decrease in the concentration gradient of the drug within the assembly relative to surrounding tissue. An added benefit is that the polymer system can help stabilize the drug. The spheres can have a size as described above for the particles.

The drug 28 can alternatively include a nanoparticle, such as a lipid nanoparticle, for delivery of molecules such as messenger ribonucleic acid (mRNA), which can be delivered in lipid nanoparticles via injection, in which particle concentration can decrease quickly. Incorporating nanoparticle delivery into the passive diffusion assembly can increase the duration during which mRNA or drug loaded into the nanoparticle would be expressed, present in the body, or a combination thereof. Nanoparticles can be delivered through porous media. Diffusion of solid particles such as nanoparticles can be increased.

In a further embodiment, the drug may be suspended or dissolved in a non-polar phase, such as liquid paraffin, a lipid or lipid mixture, or synthetic or natural oil, where the non-polar phase is immiscible or only partially miscible in water, and where the non-polar phase is selected to maintain stability of the drug over 3 or 6 or 12 months. The non-polar phase may be present in within entire interior of the drug-delivery device, or be initially only present in a single compartment of the drug-delivery device.

In a further embodiment, the drug is initially only present in the non-polar phase in a first compartment of a two-compartment drug-delivery device and the compartments are separated by a first porous membrane having properties selected to deliver the drug by diffusion from the first compartment to the second compartment. The second compartment is initially filled with a polar phase, miscible in water, or which may be water, and is fitted with a second porous membrane that further discharges the drug by diffusion into the surrounding tissue. The porous properties of the membranes are selected such that drug permeability of the second porous membrane is higher than that of the first porous membrane, as indicated, such that the drug is predominantly maintained in the first compartment during the duration of the drug-delivery process.

In further embodiments, the drug suspension (i.e., comprising the excipient) housed by the drug-delivery device may comprise emulsions, colloidal suspensions, surfactants, perfluorinated compounds, and polymers including fluoropolymers and nanocellulose.

In one embodiment, a second drug formulation 29B initially provided in the second compartment 16, along with the first drug formulation 29A in the first compartment 14. The second drug formulation 29B may contain one or more of a second drug 29C and a second drug excipient 28D. Depending on the patient requirements, the second drug 29C may be the same as or different from the first drug 28, and, e.g., may be provided in a same or different type of degradable shell 29D as the first drug 28, though this example is not intended on limiting the scope of the embodiments. Similarly, the first drug 28 may be provided in a first drug concentration and the second drug 29C may be provided in a second drug concentration that is the same as or different from the first drug concentration. The first and second excipients may be the same as or different from each other. Both excipients prevent altering or otherwise denaturing of the drug 28 prior to being diffused into the area 23 of the patient.

For example, a high dose concentration of a drug may be in the first compartment, with a first excipient for stability purposes. The first drug formulation may be clinically undesirable for immediate diffusion into the patient. A different dose concentration (e.g., less) of the same or different drug may be in the second compartment in order to diffuse rapidly into the patient. In comparison, the second drug formulation may be clinically desirable for immediate diffusion into the patient. In another example, there may be a drug formulation in the first compartment and only an excipient initially in the second compartment. This situation may be desirable where, for example, the solvent in the second compartment is polar, allowing it to better mix with the bodily fluids of the patient, as a nonlimiting example. That is, as polar and non-polar fluids do not mix, the non-polar solvent in the first compartment will remain in that compartment, thereby enabling the drug to mix with the polar solvent before being exposed to the bodily fluids.

For example, the second drug 29C may be the same as the first drug 28 and the second drug concentration may be less than the first drug concentration. This may enable a rapid infusion of the first drug 28 based on the concentration in the second compartment 16 followed by a longer duration of infusion of the first drug 28 based on the concentration in the first compartment 14. Alternatively, where different drugs are required for infusion at different rates, the second drug 29C may be different from the first drug 28 to enable a more rapid infusion of the second drug 29C. In such circumstance, depending on patient requirements, the drug concentration of the second drug 29C may be the same as, greater than, or less than the drug concentration of the first drug 28.

In exemplary embodiments, the first porous membrane 24 is generally finer, having a smaller pore size relative to second porous membrane 26, although the present disclosure is not limited thereto.

However, it is noted that the mean pore size of the first porous membrane 24 can be as large as about 20 μm, or as large as about 100 μm, or possibly even larger, for example about 1 to about 100 μm.

The second porous membrane 26 is generally coarser relative to the first porous membrane 24, with the second porous membrane 26 generally having a larger pore size than the first porous membrane 24, although the present disclosure is not limited thereto.

An exemplary range of the mean pore size of the second porous membrane 26 can be between about 1 to about 100 μm, or about 1 to about 500 μm although the present disclosure is not limited thereto. In some embodiments, the mean pore size of the first porous membrane 24 can be substantially the same as or similar to the mean pore size of the second porous membrane 26.

In exemplary embodiments, the coarseness and microstructure of the second porous membrane 26 can prevent bio-fouling of assembly 10, whereas the tightness of the first porous membrane 24 can allow for precision dosage/control over diffusion/drug 28 delivery to eventual area 23.

As described above, it is also possible for all or a part of the housing to be porous. In an aspect, housing 12 can be solid at the first compartment and porous at the second compartment. Alternatively, housing 12 can be porous at the first compartment and solid at the second compartment. In an embodiment, the housing is fully porous. An exemplary range of the mean pore size of the housing 12 can be between about 1 to about 100 μm, or between about 1 to about 500 μm, and can vary, for example being finer in the housing forming the first compartment and coarser in the housing defining the second compartment. In some embodiments, a mean pore size of the first porous membrane 24, the second porous membrane 26, and all or a portion of the housing 12 can be substantially the same as or similar to each other. In other exemplary embodiments, the coarseness and microstructure of the second porous membrane 26 and all or a portion of the housing 12 can prevent bio-fouling of assembly 10, whereas the fineness of the first porous membrane 24 can allow for precision dosage/control over diffusion/drug 28 delivery to eventual area 23.

In non-limiting examples, each porous membrane 24, 26 utilized to regulate the mass transfer of the drug 28 can be fabricated from a biocompatible metal (e.g., magnesium, zinc, titanium, iron, stainless steel, or an alloy thereof). Each porous membrane 24, 26 can also be fabricated from a biocompatible polymer, e.g., a poly(meth) acrylate or copolymer thereof, a polyurethane, a polyether ketone, or the like; or a biodegradable polymer (e.g., a polyester such as a polycaprolactone, polylactide (e.g., PLA or PLLA, etc.), a polyglycolic acid, or a copolymer thereof, a polysaccharide, or the like. Where the Each porous membrane 24, 26 is biodegradable, it is preferably selected to have a slower biodegradability than any biodegradable substance associated with drug 28, as described above. Each porous membrane 24, 26 can be in the shape of a flat cylinder or thin needle, with diameters between about 0.25 to about 10 mm and thicknesses between about 0.25 to about 10 mm, although the present disclosure is not limited thereto.

In general, each compartment 14, 16 is configured and dimensioned to house drug 28, e.g., in solution or as active agent particles or other slow release form.

As such, FIG. 1 depicts an exemplary drug delivery assembly 10, with the assembly 10 having first and second compartments 14, 16, and having first and second porous membranes 24, 26 to regulate the delivery of a drug or active agent particles 28 to area 23 that is external to assembly 10.

Exemplary assembly 10 includes first and second compartments 14, 16 separated by the first porous membrane 24. It is noted that the second compartment 16 has two openings (or first and second apertures 18, 22), that is, first opening 18, which may be formed in a divider wall 18A, joining the first and second compartments 14, 16, and second opening 22 in a second end wall 22A (or second closure), which is opposite a first end wall 22B (or first closure), and which is in fluid communication with area 23. In one embodiment the first porous membrane 24 is in the opening 18 in the divider wall 18A. In one embodiment the first porous membrane 24 itself forms the divider wall 18A, in which case it may exclude the first aperture 18. In one embodiment the second porous membrane 26 is in the second opening 22 in the second wall 22A. In one embodiment the second porous membrane 26 itself forms the second wall 22A, in which case it may exclude the second opening 22.

In exemplary embodiments, the assembly 10 is configured and dimensioned to be implanted in a body of a patient or the like (e.g., the tissue of a body of a patient or the like). Assembly 10 can be located in other/different areas of the body or the like. More than one assembly 10 can be used, at different locations of the body.

Exemplary assembly 10 is an improvement over other conventional designs/assemblies, as the dual compartment 14, 16 feature of assembly 10 has been shown via modeling to be able to deliver the medication 28 within therapeutic concentrations over longer periods of time to area 23, and to be able to deliver a larger percentage of the loaded dose 28 within those same concentrations. Use of slow- or fast-release technologies within the compartment can provide even finer tuning of drug release. For example, a solution comprising the drug can provide an initial faster (higher concentration) release for a first period of time, followed by a slower release over a second period of time as the drug is released within the compartment from, for example, a dissolvable gel or encapsulant. Use of a plurality of encapsulants, for example, can provide multiple release concentrations over multiple time periods. For other therapies, where initial slow release (low concentration) is desired for a first period of time, followed by faster (higher concentration) over a second period of time can be provided, for example by encapsulating the drug separately at two concentrations, one lower and one higher, or an outer shell having a slower release rate and in inner shell having higher release rate that is exposed after erosion of the outer shell.

Some conventional approaches for extended drug release include an osmotic pump and some polymer matrix based compositions that deliver medication for an extended period of time up to thirty to 120 days (polymer matrix systems), and 180 to 360 days (osmotic pump systems). The passive diffusion based drug 28 delivery of exemplary assembly 10 is able to deliver medication for 180 days or longer, for example, up to 1,220 days. Moreover, this exemplary assembly 10 can increase the percent of the initial dose delivered while extending the time spent in a therapeutic concentration window while still maintaining a small footprint.

Furthermore, the second porous membrane 26 separating the second compartment 16 from the external area 23 can be much coarser than the first porous membrane 24 of assembly 10 separating the first and second compartments 14, 16. This can allow for the retention of precise drug delivery with the finer media of membrane 24, and can prevent bio-fouling as the coarser porous membrane 26 is inherently resistant, as determined by testing, to the formation of protein films (e.g., on the second end 13 of the housing 12). In some embodiments, the first porous membrane 24 is coarser (more permeable) than the second porous membrane 26, depending, e.g., on how quickly the drug is intended to reach the patient. A rapid dispensing to a patient may be desired if the drug in the second compartment is not stably stored there, due to the nature of the applied excipient in that compartment. For example, if the drug were stably stored in the first compartment, due to the excipient in that compartment, it may be stored there for an extended period of time, depending on the membrane utilized between the compartments. Then, as the drug diffuses to the second compartment, there may be a desire to have it diffuse to the patient more rapidly because of the different excipient utilized in the second compartment. For that reason, a more permeable membrane may be placed at the outlet of the assembly. It is to be appreciated that various techniques can provide the greater or lesser permeability in the different porous members, and coarseness is one of several nonlimiting options in obtaining the desired permeability.

In an example embodiment, the internal volume of housing 12 can be about 1 mL, with the internal volume divided into a 0.6 mL compartment in first compartment 14, and with a 0.4 mL compartment in second compartment 16. It is noted that a total internal volume of the first compartment and of the second compartment (added together) can range from about 0.10 mL to 5 mL, preferably between 0.10 to 2 mL.

The overall diameter of housing 12 of assembly 10 can be about 7 mm, and the length of housing 12 of assembly 10 can be about 4 cm. Use of extended drug delivery mechanisms as described above, in particular dissolvable gels or a solid support, can result in larger dimensions. The overall diameter of housing 12 of assembly 10 can be about 7 mm, and the length of housing 12 of assembly 10 can be about 4 cm.

In an example embodiment, the second porous membrane 26 can be classified as a Media Grade (“MG”) 0.1 having a mean pore size of about 0.1 μm. The diameter D3 of this exemplary second porous membrane 26 can be about 0.5 mm by about 1 mm thick. The first porous membrane 24 can be classified as a MG 2, with a diameter D2 of about 1.5 mm by about 3 mm thick. The first and second membranes 24, 26 can be fabricated out of 99.99% pure zinc, although the present disclosure is not limited thereto. It is to be appreciated that the diameter is another variable in controlling the mass transport rate of the drug through the membrane. Thus, the relative sizes of the diameters of the membranes as shown and discussed is not intended on limiting the scope of the embodiments.

In use, exemplary assembly 10 and the other embodiments described herein can be utilized for extended delivery of drug 28, which includes any therapeutic or active agent such as a medication, pharmaceutical, supplement, or the like, or a reporting compound (e.g., a radiotracer or fluorescent compound) for monitoring drug delivery or other biological process. The drug can be a biologic, a protein, a pharmaceutical or reporter small molecules (10 to 1,000 g/mol), or a combination thereof. In an embodiment, the drug is a biologic. Preferably, the sustained release of the drug 28 is for a period of greater than six months, for example more than 6 months to two years or more than 6 months to one year. This can overcome difficulties with daily, weekly, or monthly dosing regimens.

As indicated above, the second drug formulation 29B may be the same as or different from the first drug formulation 29A. As non-limiting examples, the second drug formulation 29B may include the second drug 29C and the second excipient 28D. As further nonlimiting examples, the second drug 29C may be provided in a particulate form, where the second drug 29C is formed as a microparticle or a nanoparticle. The second drug 29C may be encapsulated in a degradable shell, where the degradable shell may be formed of a polymer or a gel. The second excipient 28D may include an emulsion, a colloidal suspension, a surfactant, a perfluorinated compound, and a polymer including a fluoropolymer and nanocellulose.

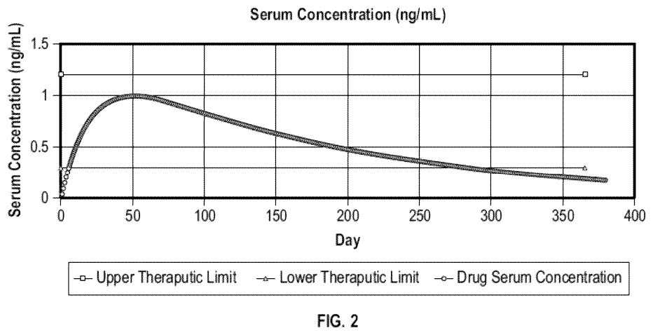

In FIG. 2, the release profile of a drug 28 through an exemplary assembly 10 is shown. For this example, the drug 28 was only loaded in the first compartment 14 (behind the first membrane 24), but the drug 28 could be loaded in both compartments 14, 16 theoretically. In FIG. 2, the y-axis details serum concentration, or a description of how concentrated the drug 28 is predicted to be in the blood after x days. The orange and gray lines detail a concentration window where the serum concentration indicates that the drug is effective. The exemplary assembly 10 having compartments 14, 16 allows for an extension of the time spent in this window by better regulating the diffusion gradient between the second compartment 16 and the area 23 by introducing an intermediate volume (compartment 16) and membrane 24 to facilitate mass transfer.

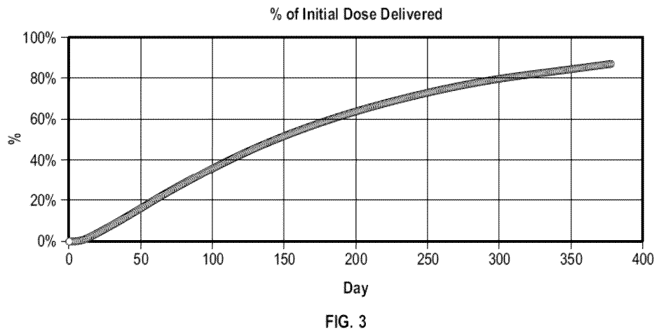

In FIG. 3, the predicted percent released over time relationship for an assembly 10 having compartments 14, 16 is shown. A goal of a drug delivery assembly is to have the percent released close to 90% or greater at the time which the serum concentration is modeled to be below the minimum effective therapeutic level. An assembly 10 having compartments 14, 16, via a better control over the drug delivery physics described above, allows for the achievement of a higher percentage delivered at this time.

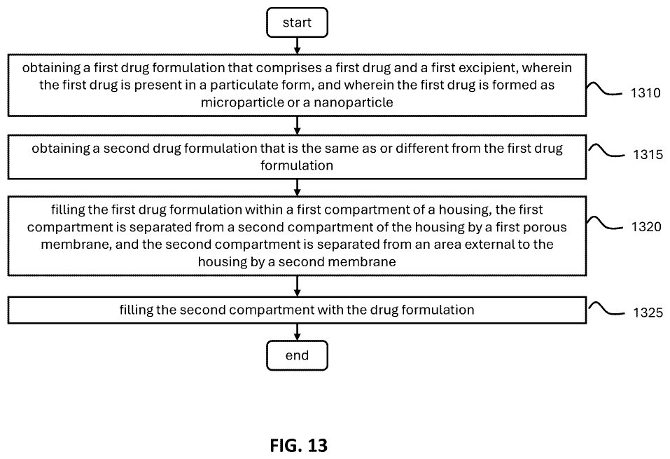

Turning to FIG. 13, a flowchart shows a method of preparing the drug delivery assembly 10, e.g., prior to implanting in a patient. Refilling the assembly 10 after implanting according to the disclosed method is within the scope of the embodiments. In FIG. 13, boxes in dashed lines in the flowchart represent further explanations, including alternative embodiments, of one or more preceding steps and are not intended to limit the scope of the embodiments.

As shown in block 1310, the method includes obtaining the first drug formulation 29A that includes the first drug 28 and the first excipient 28C. As indicated, the first drug 28 is present in a particulate form, and the first drug 28 is formed as microparticle or a nanoparticle. As shown in block 1315, the method includes obtaining a second drug formulation 29B that is the same as or different from the first drug formulation 29A. As shown in block 1320, the method includes filling the first drug formulation 29A within the first compartment 14 of the housing 10. As indicated, the first compartment 14 is separated from the second compartment 16 by the first porous membrane 18, and the second compartment 16 is separated from an area 23 external to the housing 10 by the second membrane 22. As shown in block 1325, the method includes filling the second compartment 16 with the second drug formulation 29B. As indicated, the second drug formulation 29B includes one or more of the second drug 29C and the second excipient 28D. The second drug 29C may be the same as or different from the first drug 28. The second excipient 29D may be the same as or different from the first excipient 28C. The first drug 28 may be provided in a first drug concentration in the first drug formulation 29A, and the second drug 29C may be provided in a second drug concentration in the second drug formulation 29B. The second drug concentration may be the same as or different from the first drug concentration.

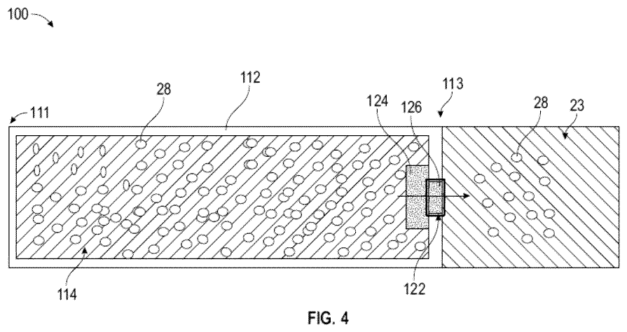

In another embodiment and as shown in FIG. 4, exemplary drug delivery assembly 100 takes the form of a single compartment and dual porous membrane based (e.g., porous zinc membrane based) drug delivery assembly 100 for extended drug 28 delivery (e.g., via passive diffusion) and/or tunability or the like.

Exemplary drug delivery assembly 100 includes a housing 112 that extends from a first end 111 to a second end 113. In exemplary embodiments, the housing 112 is substantially tubular or substantially cylindrical, although the present disclosure is not limited thereto. Rather, the housing 112 can take a variety of shapes and/or forms.

Housing 112 can be fabricated from a variety of materials as described above. For example, housing 112 can be fabricated from a metal (e.g., magnesium, zinc, iron, stainless steel, or an alloy thereof). Housing 112 can also be fabricated from a biodegradable plastic (e.g., PLA or PLLA, etc.), a polyglycolide, a polysaccharide, or the like.

In non-limiting examples, housing 112 takes the form of a tube (cylindrical) shape, as similarly discussed above relative to housing 12 of assembly 10.

Exemplary housing 112 defines a first compartment 114. An opening 122 in housing 112 is positioned at the second end 113 of housing 112 of assembly 100, as shown in FIG. 4. In general, opening 122 can be in communication with an area 23 that is external to the housing 112 of assembly 100 (e.g., an area 23 such as, for example, the surrounding tissue or other location in a of a body of a patient, or test subject, after assembly 100 is positioned in the body).

In exemplary embodiments, a first porous membrane 124 is positioned proximal to the opening 122, and a second porous membrane 126 is positioned substantially within the opening 122, as discussed further below.

It is noted that the first porous membrane 124 can be attached and/or bonded relative to the opening 122 of housing 112 (and/or to membrane 126) and that the second porous membrane 126 can be attached and/or bonded relative to the opening 122 of housing 112 (and/or to membrane 124) via various attachment or bonding methods (e.g., sintering bonding, adhesive, press-fit, etc.).

Interior cavity of first compartment 114 can be filled or partially filled with a drug 28 as described above.

In exemplary embodiments, the first porous membrane 124 is generally finer, having a smaller pore size relative to second porous membrane 126. In certain embodiments, the first porous membrane 124 has a smaller mean pore size than the second porous membrane 126.

An exemplary range of the mean pore size of the first porous membrane 124 can be between about 0.05 to about 1.0 μm. The second porous membrane 126 is generally coarser relative to the first porous membrane 124, with the second porous membrane 126 generally having a larger pore size than the first porous membrane 124.

An exemplary range of the mean pore size of the second porous membrane 126 can be between about 1 to about 100 μm, although the present disclosure is not limited thereto.

In exemplary embodiments, these two membranes 124, 126, are joined or attached together to form one continuous body, leaving a porous matrix 124 and 126 with a gradient pore size structure. In an embodiment, the two membranes are a single membrane having a porous gradient (stepwise or continuous) of coarser to finer porosities from one surface to the opposite surface.

In exemplary embodiments, the coarseness (larger pores) of the second porous membrane 126 can prevent bio-fouling of assembly 100, whereas the tightness of the first porous membrane 124 can allow for precision dosage/control over drug 28 delivery to external area 23.

In non-limiting examples, each porous membrane 124, 126 utilized to regulate the mass transfer of the drug 28 can be fabricated from zinc, titanium, a polymer, for example polyether ether ketone, or another applicable or suitable biomaterial as described for the porous membranes 24, 26. Each porous membrane 124, 126 can be in the shape of a flat cylinder or thin needle, with diameters between about 0.25 to about 5 mm and thicknesses between about 0.25 to about 10 mm, although the present disclosure is not limited thereto.

In general, compartment 114 is configured and dimensioned to house drug, e.g., whether in solution, as particles, or other form as described above.

As such, FIG. 4 depicts an exemplary drug delivery assembly 100, with the assembly 100 having compartment 114, and having first and second porous membranes 124, 126 to regulate the delivery of drug 28 to area 23 that is external to assembly 100.

In exemplary embodiments, it is noted that the assembly 100 is configured and dimensioned to be implanted in a body of a patient, test subject, or the like (e.g., the tissue of a body of a patient, test subject, or the like). It is also noted that the assembly 100 can be located in other/different areas of the body or the like as described above. More than one assembly 100 can be used in different locations of the body.

In use, exemplary assembly 100 can be utilized for the extended (sustained) delivery of drug 28. Preferably, the sustained release of the drug 28 is for a period of greater than six months, for example more than 6 months to two years or more than 6 months to one year. This can overcome difficulties with daily, weekly, or monthly dosing regimens.

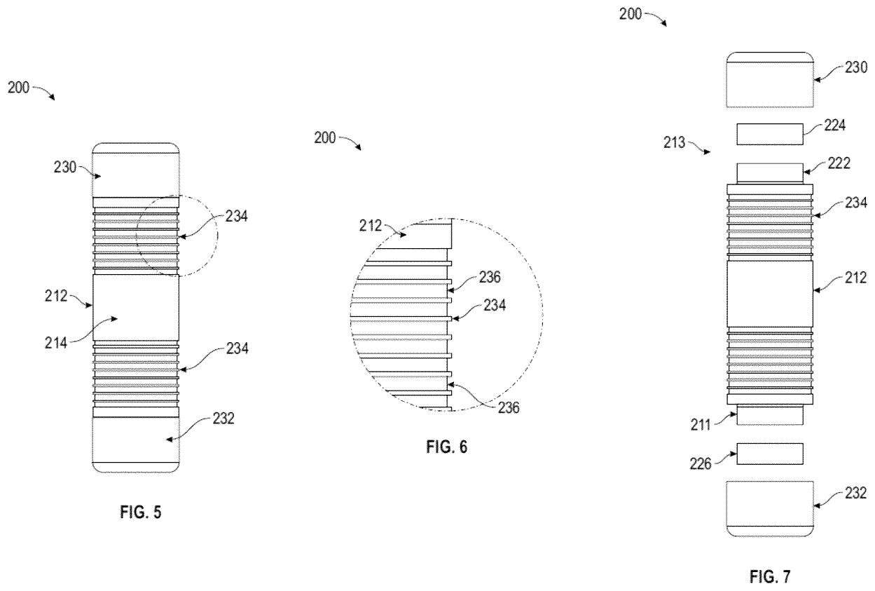

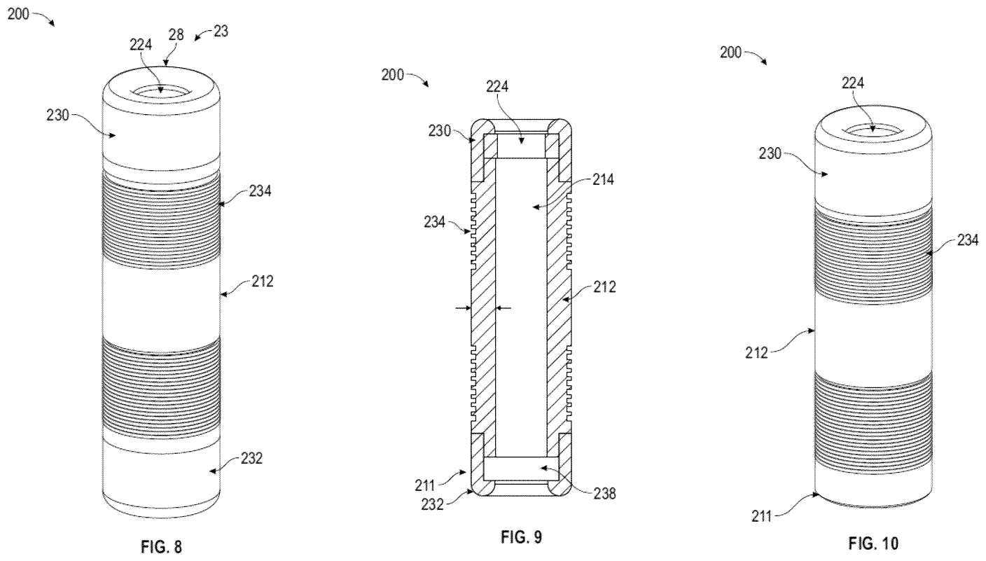

With reference to one or more of FIGS. 5-12, other embodiments of exemplary drug delivery assembly 200 includes a housing 212 that extends from a first end 211 to a second end 213. In exemplary embodiments, the housing 212 is substantially tubular or substantially cylindrical with a hollow interior, although the present disclosure is not limited thereto. Rather, it is noted that housing 212 can take a variety of shapes and/or forms.

It is noted that housing 212 can be fabricated from a variety of materials as described above. For example, housing 212 can be fabricated from a metal (e.g., magnesium, zinc, titanium, iron, stainless steel, or an alloy thereof). Housing 212 can also be fabricated from a biodegradable polymer (e.g., PLA or PLLA, etc.).

In non-limiting examples, housing 212 takes the form of a tube or substantially cylindrical shape, as similarly discussed above. In exemplary embodiments, the housing 212 can extend about 1.02 cm or about 1.29 cm or about 1.63 cm from first end 211 to second end 213. In exemplary embodiments, the housing 212 can have a wall thickness of about 0.1016 cm.

Exemplary housing 212 defines a first compartment 214 shown explicitly in FIGS. 9 and 12. The interior cavity of the first compartment 214 can be fully or partially filled with a drug 28 as discussed above.

In certain embodiments, a first porous membrane 224 is positioned proximal to the opening 222, and a second porous membrane 226 is positioned proximal to first end 211 as shown in FIG. 7, or distant from first end 211, as discussed further below.

An opening 222 in housing 212 is positioned at the second end 213 of housing 212 of assembly 200. In general, opening 222 can be in communication with an area that is external to the housing 212 of assembly 200 (e.g., an area such as, for example, the surrounding tissue of a body of a patient or test subject), after assembly 200 is positioned in the body). In exemplary and non-limiting embodiments, the compartment can have a total internal volume of about 0.25 ml or about 0.50 ml or about 1.00 ml.

In some embodiments, the first porous membrane 224 can be attached and/or secured relative to the opening 222 of housing 212 via a first end cap (or first closure) 230 attached and/or secured to second end 213, and the second porous membrane 226 (if adjacent the first end 211) can be attached and/or secured relative to the first end 211 of housing 212 via a second end cap (or second closure) 232 attached and/or secured to first end 211. It is noted that first end cap 230 can include a hood-like feature that is designed to prevent fibrosis from impeding the diffusion properties of the first porous membrane 224. In exemplary embodiments, the first end cap 230 can have an outer diameter of about 0.34 cm or about 0.43 cm or about 0.54 cm.

An exemplary range of the mean pore size of the first and second porous membranes 224, 226 can each be between about 0.10 to about 100 μm, although the present disclosure is not limited thereto.

In exemplary embodiments, each porous membrane 224, 226 (utilized to regulate the mass transfer of the drug 28) can be fabricated from a material as described above, for example from zinc, titanium, polyether ether ketone, or another applicable or suitable biomaterial. Each porous membrane 224, 226 can be in the shape of a flat cylinder or thin needle, with diameters between about 0.25 to about 10 mm and thicknesses between about 0.25 to about 10 mm, although the present disclosure is not limited thereto.

Further with reference to FIGS. 5-12, the housing 211 includes at least one grooved or threaded section 234 (e.g., two or more sections 234). Such grooved or threaded section can optionally be present in any of the embodiments discussed herein. Each grooved or threaded section 234 includes a plurality of tissue grooves 236 (see, e.g., FIG. 6). As such, the outside surface of the housing 212 is lined with grooved or threaded sections 234 having a plurality of tissue grooves 236, with the tissue grooves 236 promoting the adhesion of tissue to the assembly 200 (e.g., to prevent assembly 200 migration within the body). The grooved or threaded sections 234 modify the surface roughness of the exterior of the assembly 200 to promote tissue growth around the assembly 200 to hold it in place. As such, the grooved or threaded sections 234 along housing 212 act to promote tissue adhesion and reduce implant assembly 200 migration. In an embodiment, the thread feature 234 conforms to a #6-40 thread classified by ASME B1.1.

Features such as hooks/loops can be added to housing 212 (or housing 112) to promote suturing of the assembly 200 (e.g., to a piece of the dermal layer). Additionally, polymeric coatings can be applied to housing 212 (or housing 114) to modify the chemical and/or physical properties at the surface of the housing 212 of assembly 200. It is noted that a large range of surface roughening processes, such as various types of threading, sanding, chemical etching, sand blasting, and other modification methods can also be utilized on housing 212 (or 112) to promote the adhesion of tissue to the assembly 200. Preferably, raised surface features in the range of 200 to 500 microns can be created on the surface of the housing 212 to promote adhesion of tissue to the assembly 200.

It is noted that an entirely dissolvable design of housing 112 or 212 can be fabricated from zinc, and a refillable design of housing 112 or 212 can be fabricated from titanium, although the present disclosure is not limited thereto.

FIGS. 5-8 depict an embodiment of an exemplary drug delivery assembly 200, with the assembly 200 defining a first compartment having first and second porous membranes 224, 226 to regulate the delivery of a drug 28 to area 23 that is external to assembly 200.

In an embodiment, the second porous membrane 226 can be attached to a filling apparatus or the like in order to fill compartment 214 with drug 28, either before or after assembly 200 is implanted. The second porous membrane 226 can be closed after filling compartment 214 with drug 28 (e.g., closed with a plug of hydroxyapatite cement or other biocompatible cement).

FIG. 9 depicts another exemplary drug delivery assembly 200, with the assembly 200 having compartment 214, and having first porous membrane 224 secured to housing 212 via first end cap 230, and with a septum member 238 secured to first end 211 via second end cap 232. The septum member 238 can be attached to a filling apparatus or the like in order to fill compartment 214 with drug 28, either before or after assembly 200 is implanted. The septum member 238 can be closed, if desired, after filling compartment 214 with drug (e.g., closed with a plug of hydroxyapatite cement or other biocompatible cement, or a solid cap 232 having no opening therein).

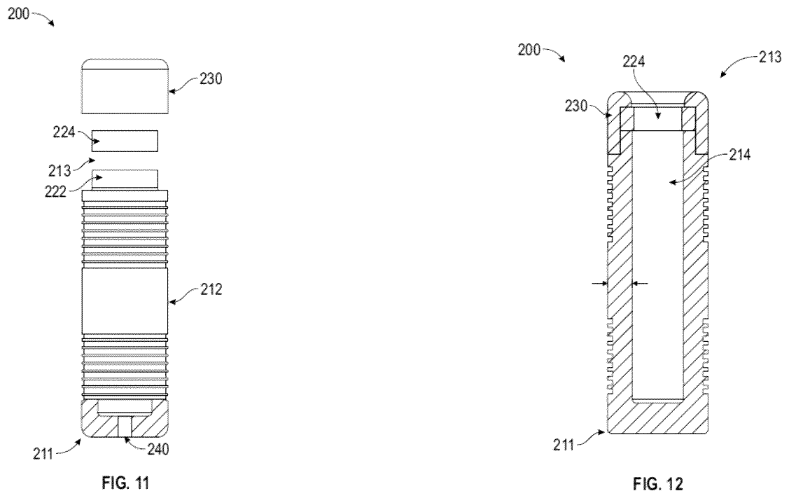

FIGS. 10-11 depict an exemplary drug delivery assembly 200, with the assembly 200 having compartment 214, and having first porous membrane 224 secured to housing 212 via first end cap 230, and with a drug loading port 240 positioned at first end 211. The drug loading port 240 can be attached to a filling apparatus or the like in order to fill compartment 214 with drug 28, either before or after assembly 200 is implanted. The drug loading port 240 can be closed, if desired, after filling compartment 214 with drug 28 (e.g., closed with a hydroxyapatite cement or other biocompatible cement).

FIG. 12 depicts an exemplary drug delivery assembly 200, with the assembly 200 having compartment 214, and having first porous membrane 224 secured to housing 212 via first end cap 230, and with first end 211 being closed off. The compartment 214 can be filled or partially filled with drug 28 via second end 213 (e.g., before membrane 224 is secured to housing 212).

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. “About” as used herein means the usual error range for measurement of the respective value readily known to the skilled person in this technical field, for example ±1%, or ±2%, or ±5% of the stated value. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Although the systems and methods of the present disclosure have been described with reference to exemplary embodiments thereof, the present disclosure is not limited to such exemplary embodiments and/or implementations. Rather, the systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

What is claimed is:

1. A drug delivery assembly, comprising:
a housing that extends from a first end to a second end,
wherein the housing defines an interior space, with a first closure at the first end, a second closure at the second end, and a divider wall between the first end and the second end, such that the housing further defines:
a first compartment located at the first end of the housing, between the first closure and the divider wall, and
a second compartment located at the second end of the housing between the divider wall and the second closure,
wherein:
the divider wall comprises a first porous membrane, or defines a first aperture that includes the first porous membrane, wherein the first porous membrane provides fluid communication between the first compartment and the second compartment; and
the second closure comprises a second porous membrane, or defines a second aperture that includes the second porous membrane, wherein the second porous membrane provides fluid communication between the second compartment and an area that is external to the housing; and
a first drug formulation disposed within the first compartment, the first drug formulation comprising a first drug and a first excipient, and the second compartment includes a second drug formulation that is the same as or different from the first drug formulation.

2. The assembly of claim 1, wherein:
the first drug is present in a particulate form, and wherein the first drug is formed as a microparticle or a nanoparticle.

3. The assembly of claim 1, wherein:
the first porous membrane has a lower drug permeability than the second porous membrane.

4. The assembly of claim 1, wherein:
the first drug is encapsulated in a degradable shell that is formed of a polymer or a gel.

5. The assembly of claim 1, wherein:
the first drug formulation comprises a non-polar compound immiscible in water; and
the second compartment includes a polar solvent miscible in water.

6. The assembly of claim 5, wherein:
the non-polar compound is one or more of: liquid paraffin; a lipid; synthetic oil; or
natural oil; or
the polar solvent is water.

7. The assembly of claim 1, wherein:
the first excipient comprises one or more of: an emulsion; a colloidal suspension; a surfactant; a perfluorinated compound; and a polymer including a fluoropolymer and nanocellulose.

8. The assembly of claim 1, wherein:
the second drug formulation comprises one or more of a second drug and a second excipient, wherein the second drug is the same as or different from the first drug, and the second excipient is the same as or different from the first excipient; and
wherein the first drug is provided in a first drug concentration in the first drug formulation and the second drug is provided in a second drug concentration in the second drug formulation, wherein the second drug concentration is the same as or different from the first drug concentration.

9. The assembly of claim 8, wherein:
the second drug is the same as the first drug; and
the second drug concentration is less than the first drug concentration.

10. The assembly of claim 1, wherein the second drug formulation comprises one or more of:
a second drug that comprises one or more of:
a particulate form, and wherein the second drug is formed as a microparticle or a nanoparticle; or
a degradable shell in which the second drug is encapsulated, wherein the degradable shell is formed of a polymer or a gel; or
a second excipient that comprises one or more of:
an emulsion; a colloidal suspension; a surfactant; a perfluorinated compound; or a polymer including a fluoropolymer and nanocellulose.

11. A method of preparing the drug delivery assembly, comprising:
obtaining a first drug formulation that comprises a first drug and a first excipient, wherein the first drug is present in a particulate form;
obtaining a second drug formulation that is the same as or different from the first drug formulation;
filling the first drug formulation within a first compartment of a housing, wherein the first compartment is separated from a second compartment of the housing by a first porous membrane, and the second compartment is separated from an area external to the housing by a second membrane; and
filling the second compartment with the second drug formulation,
wherein:
the housing extends from a first end to a second end, and defines an interior space, with a first closure at the first end, a second closure at the second end, and a divider wall between the first end and the second end,
wherein that the housing defines:
the first compartment located at the first end of the housing, between the first closure and the divider wall, and
the second compartment located at the second end of the housing between the divider wall and the second closure, and
wherein:
the divider wall comprises the first porous membrane, or defines a first aperture that includes the first porous membrane, wherein the first porous membrane provides fluid communication between the first compartment and the second compartment; and
the second closure comprises the second porous membrane, or defines a second aperture that includes the second porous membrane, wherein the second porous membrane provides fluid communication between the second compartment and an area that is external to the housing.

12. The method of claim 11, wherein:
the first drug is formed as a microparticle or a nanoparticle.

13. The method of claim 11, wherein:
the first porous membrane has a lower drug permeability than the second porous membrane.

14. The method of claim 11, wherein:
the first drug is encapsulated in a degradable shell that is formed of a polymer or a gel.

15. The method of claim 11, wherein:
the first drug formulation comprises a non-polar compound immiscible in water; and
the second compartment includes a polar solvent miscible in water.

16. The method of claim 15, wherein:
the non-polar compound is one or more of: liquid paraffin; a lipid; synthetic oil; or natural oil; or
the polar solvent is water.

17. The method of claim 11, wherein:
the first excipient comprises one or more of: an emulsion; a colloidal suspension; a surfactant; a perfluorinated compound; and a polymer including a fluoropolymer and nanocellulose.

18. The method of claim 11, wherein:
the second drug formulation comprises one or more of a second drug and a second excipient, wherein the second drug is the same as or different from the first drug, and the second excipient is the same as or different from the first excipient; and
the first drug is provided in a first drug concentration in the first drug formulation and the second drug is provided in a second drug concentration in the second drug formulation, wherein the second drug concentration is the same as or different from the first drug concentration.

19. The method of claim 18, wherein:
the second drug is the same as the first drug; and
the second drug concentration is less than the first drug concentration.

20. The method of claim 11, wherein the second drug formulation comprises one or more of:
a second drug that comprises one or more of:
a particulate form, wherein the second drug is formed as a microparticle or a nanoparticle; or
a degradable shell in which the second drug is encapsulated, wherein the degradable shell is formed of a polymer or a gel; or
a second excipient that comprises one or more of:
an emulsion; a colloidal suspension; a surfactant; a perfluorinated compound; or a polymer including a fluoropolymer and nanocellulose.
